(19) United States
(12) Patent Application Publication
(10) Pub. No.: US 2024/0315952 A1
(43) Pub. Date: Sep. 26, 2024

(54) PERSONAL CARE COMPOSITION

(71) Applicant: Conopco, Inc., d/b/a UNILEVER, Englewood Cliffs, NJ (US)

(72) Inventors: Xuelan GU, Shanghai (CN); Tingyan MI, Shanghai (CN)

(73) Assignee: Conopco, Inc., d/b/a UNILEVER, Englewood Cliffs, NJ (US)

(21) Appl. No.: 18/269,330

(22) PCT Filed: Dec. 6, 2021
(86) PCT No.: PCT/EP2021/084301

§ 371 Date: Jun. 23, 2023

(30) Foreign Application Priority Data
Dec. 25, 2020 (WO) PCT/CN2020/139518
Jan. 28, 2021 (EP) 21153895.4

(51) Int. Cl. A61K 8/9789; A61K 8/67; A61K 31/455; A61K 36/185; A61P 29/00; A61Q 19/00

(52) U.S. Cl. CPC A61K 8/9789; A61K 8/675; A61K 31/455; A61K 36/185; A61K 36/5777; A61P 29/00; A61Q 19/00; A61K 2800/10

(57) ABSTRACT

Disclosed is a personal care composition comprising extract of moringa: extract of plant of genus Theobroma; and 0.00001 to 0.08% of vitamin B3 compound by weight of the composition.

### FIELD OF THE INVENTION

The present invention relates to a personal care composition comprising extract of moringa; extract of plant of genus Theobroma; and 0.00001 to 0.08% of vitamin B3 compound by weight of the composition. In particular such composition is capable of inhibiting release of pro-inflammatory cytokines generated due to contact of skin with pollutants.

### BACKGROUND OF THE INVENTION

Air pollution is a big problem, particularly in some of the developing countries. Particulate matter (PM) is one of the important to affect the air quality which are inhalable particles composed of sulphate, nitrates, ammonia, sodium chloride, black carbon, mineral dust and water. Particles with a diameter of less than 10 microns (PM10), including fine particles less than 2.5 microns (PM2.5) pose the greatest risks to health, as they can enter the lungs and the bloodstream. In addition, PM may bring adverse effects of pollution on human skin. These adverse effects include premature ageing, development of fine lines and wrinkles, pigmented spots, hyperpigmentation, rash and inflammation.

PM induces oxidative stress via production of reactive oxygen species and secretion of pro-inflammatory cytokines such as TNF-α, IL-1α, and IL-8. In addition, the increased production of ROS such as superoxide and hydroxyl radical by PM exposure increases MMPs including MMP-1, MMP-2, and MMP-9, resulting in degradation of collagen. These processes lead to the increased inflammatory skin diseases and skin aging. Overall, increased PM levels are associated with the development of various skin diseases via the regulation of oxidative stress and inflammatory cytokines.

Therefore, we have recognized there is a need to develop a person care composition which is able to protect the skin from harmful effects of atmospheric pollutants. The present inventors surprisingly found that a combination of extract of moringa, extract of plant of genus Theobroma, and a little amount of vitamin B3 compound exhibits an improved effect for inhibiting release of pro-inflammatory cytokine.

### SUMMARY OF THE INVENTION

In a first aspect, the present invention is directed to personal care composition comprising extract of moringa; extract of plant of genus Theobroma; and 0.00001 to 0.08% of vitamin B3 compound by weight of the composition.

In a second aspect, the present invention is directed to a method of alleviating, inhibiting and/or reducing at least one symptom selected from inflammation, gingivitis, periodontitis, dandruff and acne, comprising a step of applying the personal care composition of the present invention.

In a third aspect, the present invention is directed to use of the personal care composition of the present invention for alleviating, inhibiting and/or reducing at least one symptom selected from inflammation, gingivitis, periodontitis, dandruff and acne.

All other aspects of the present invention will more readily become apparent upon considering the detailed description and examples which follow.

### DETAILED DESCRIPTION OF THE INVENTION

Except in the examples, or where otherwise explicitly indicated, all numbers in this description indicating amounts of material or conditions of reaction, physical properties of materials and/or use may optionally be understood as modified by the word “about”.

All amounts are by weight of the composition, unless otherwise specified.

It should be noted that in specifying any range of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

The composition of the present invention comprises extract of moringa. Typically, the extracts of moringa are the extracts of seeds, fruits, leaves and/or bark of moringa, preferably extracts of seeds and/or fruits of moringa, and more preferably extract of seeds of moringa. Preferably, the moringa is selected from Moringa oleifera Lam., Moringa pterygosperma, Moringa peregrina, Moringa concanensis and Moringa drouhardii. More preferably, the extract of moringa is selected from extract of seeds of Moringa oleifera Lam. and Moringa pterygosperma. Most preferably, the extract of moringa is selected from extract having INCI name of Moringa Pterygosperma Seed Extract, Moringa Oleifera Seed Oil, or a combination thereof. Preferably the Moringa Pterygosperma Seed Extract is protein extracted from seeds of a Moringa oleifera.

Preferably, the extract of moringa aqueous extract of moringa. Aqueous extract refers to that the exact is obtained by using water, or water mixed with remaining solvent. The remaining solvent may be selected from ethanol, acetone, ethyl acetate, glycerin, butylene glycol or a mixture thereof. Preferably, the aqueous extract is extract obtained by using water and/or glycerin.

Preferably, the extract of moringa is present in the composition in an amount of 0.000001% to 2% by weight of the composition, more preferably in an amount of 0.00001% to 0.5%, even more preferably from 0.0003% to 0.2% and most preferably in an amount of 0.002% to 0.1% by weight of the composition.

The composition of the present invention comprises extract of plant of genus Theobroma. Typically, the extracts of plant of genus Theobroma are the extracts of seeds, fruits, leaves and/or bark of plant of genus Theobroma, preferably comprise extracts of seeds and/or fruits of plant of genus Theobroma, and more preferably comprise extract of seeds of plant of genus Theobroma. Preferably, the plant of genus Theobroma is cocoa and/or cupuagu, more preferably the plant of genus Theobroma is cocoa. Even more preferably, the extract of plant of genus Theobroma is selected from Theobroma Cacao (Cocoa) Husk Extract, Theobroma Cacao (Cocoa) Seed Extract, or a mixture thereof. Most preferably, the extract of plant of genus Theobroma is Theobroma Cacao (Cocoa) Seed Extract.

Preferably, the extract of moringa aqueous extract of plant of genus Theobrom. Aqueous extract refers to that the exact is obtained by using water, or water mixing with remaining solvent. The remaining solvent may be selected from ethanol, acetone, ethyl acetate, glycerin, butylene glycol or a mixture thereof. Preferably, the aqueous extract is extract obtained by using water and/or butylene glycol.

Preferably, the extract of plant of genus Theobroma is present in the composition in an amount of 0.000001% to 1% by weight of the composition, more preferably in an amount of 0.000004% to 0.1%, even more preferably from 0.00001% to 0.005% and most preferably in an amount of 0.00002% to 0.001% by weight of the composition.

For sake of clarity, the weight of the extract in the present invention typically refers to the weight of all ingredients extracted from the plant, excluding the weight of the extract solvent.

To achieve an improved anti-inflammatory benefit and/or suitable sensory, the weight ratio of the extract of moringa to the extract of plant of genus Theobroma is preferably in the range of 1:1 to 30000:1, more preferably 7:1 to 5000:1, and even more preferably 40:1 to 1000:1. Preferably the weight ratio of the protein in the extract of moringa to the extract of plant of genus Theobroma is preferably in the range of 1:100 to 100:1, more preferably 20:1 to 1:20, and more preferably 1:5 to 5:1.

Vitamin B3 compound as used herein also comprises niacin, niacinamide, nicotinyl alcohol, and derivatives and salts of these compounds. Derivatives of the vitamin B3 compounds typically comprise nicotinic acid esters, nicotinyl alcohol esters of carboxylic acids, niacinamide N-oxide, nicotinyl amino acids and nicotinic acid n-oxide. Preferred vitamin B3 compound is selected from niacin and niacinamide, and the particularly preferred vitamin B3 compound is niacinamide.

Preferably, the vitamin B3 compound is present in an amount of 0.00001 to 0.08% by weight of the composition, more preferably in an amount of 0.0001% to 0.06%, even more preferably from 0.0005 to 0.04% and most preferably 0.002% to 0.03% by weight of the composition.

To achieve an improved anti-inflammatory benefit and/or suitable sensory, the weight ratio of the extract of moringa to the niacinamide is preferably in the range of 1:100 to 300:1, preferably 1:30 to 100:1, and more preferably 1:10 to 30:1.

The composition preferably comprises a cleansing surfactant. More than one cleansing surfactant may be included in the composition. The cleaning surfactant may be chosen from soap, non-soap anionic, cationic, non-ionic, amphoteric surfactant and mixtures thereof. Many suitable surface-active compounds are available and are fully described in the literature, for example, in “Surface-Active Agents and Detergents”, Volumes I and II, by Schwartz, Perry and Berch. The preferred surface-active compounds that can be used are soaps, non-soap anionic, non-ionic surfactant, amphoteric surfactant or a mixture thereof.

Suitable non-soap anionic surfactants include linear alkylbenzene sulphonate, primary and secondary alkyl sulphates, particularly C₈ to C₁₅ primary alkyl sulphates; alkyl ether sulphates; olefin sulphonates; alkyl xylene sulphonates; dialkyl sulphosuccinates; fatty acid ester sulphonates; or a mixture thereof. Sodium salts are generally preferred.

Most preferred non-soap anionic surfactant are linear alkylbenzene sulphonate, particularly linear alkylbenzene sulphonates having an alkyl chain length of from C₈ to C₁₅. It is preferred if the level of linear alkylbenzene sulphonate is from 0 wt % to 30 wt %, more preferably from 1 wt % to 25 wt %, most preferably from 2 wt % to 15 wt %, by weight of the total composition.

Nonionic surfactants that may be used include the primary and secondary alcohol ethoxylates, especially the C₈ to C₂₀ aliphatic alcohols ethoxylated with an average of from 1 to 20 moles of ethylene oxide per mole of alcohol, and more especially the C₁₀ to C₁₅ primary and secondary aliphatic alcohols ethoxylated with an average of from 1 to 10 moles of ethylene oxide per mole of alcohol. Non ethoxylated nonionic surfactants include alkylpolyglycosides, glycerol monoethers, and polyhydroxyamides (glucamide). It is preferred if the level of non-ionic surfactant is from 0 wt % to 30 wt %, preferably from 1 wt % to 25 wt %, most preferably from 2 wt % to 15 wt %, by weight of a fully formulated composition comprising the microcapsules of the invention.

Suitable amphoteric surfactants preferably are betaine surfactants. Examples of suitable amphoteric surfactants include, but are not limited to, alkyl betaines, alkylamido betaines, alkyl sulfobetaines, alkyl sultaines and alkylamido sultaines; preferably, those having 8 to about 18 carbons in the alkyl and acyl group. It is preferred that the amount of the amphoteric surfactant is 0 to 20 wt %, more preferably from 1 to 10 wt %, by weight of the composition.

It is also possible to include certain mono-alkyl cationic surfactants. Cationic surfactants that may be used include quaternary ammonium salts of the general formula R¹R²R³R⁴N⁺X⁻ wherein the R groups are long or short hydrocarbon chains, typically alkyl, hydroxyalkyl or ethoxylated alkyl groups, and X is a counter-ion (for example, compounds in which R¹ is a C₈-C₂₂ alkyl group, preferably a C₈-C₁₀ or C₁₂-C₁₄ alkyl group, R² is a methyl group, and R³ and R⁴, which may be the same or different, are methyl or hydroxyethyl groups); and cationic esters (for example, choline esters).

Water-insoluble skin benefit agents may also be formulated into the compositions as conditioners and moisturizers. Examples include silicone oils; hydrocarbons such as liquid paraffins, petrolatum, microcrystalline wax, and mineral oil; and vegetable triglycerides such as sunflower seed and cottonseed oils.

Some compositions may include thickeners. These may be selected from cellulosics, natural gums and acrylic polymers but not limited by this thickening agent types. Among the cellulosics are sodium carboxymethyl cellulose, hydroxyethyl cellulose, hydroxypropyl methylcellulose and combinations thereof. Suitable gums include xanthan, pectin, karaya, agar, alginate gums and combinations thereof. Among the acrylic thickeners are homopolymers and copolymers of acrylic and methacrylic acids including carbomers such as Carbopol 1382, Carbopol 982, Ultrez, Aqua SF-1 and Aqua SF-2 available from the Lubrizol Corporation. Amounts of thickener may range from 0.01 to 3% by weight of the active polymer (outside of solvent or water) in the compositions.

Preservatives can desirably be incorporated into the compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Particularly preferred preservatives are phenoxyethanol, methyl paraben, propyl paraben, imidazolidinyl urea, sodium dehydroacetate and benzyl alcohol. The preservatives should be selected having regard for the use of the composition and possible incompatabilities between the preservatives and other ingredients. Preservatives are preferably employed in amounts ranging from 0.01% to 2% by weight of the composition.

A variety of other optional materials may be formulated into the compositions. These may include: antimicrobials such as 2-hydroxy-4,2′,4′-trichlorodiphenylether (triclosan), 2,6-dimethyl-4-hydroxychlorobenzene, and 3,4,4′-trichlorocarbanilide; scrub and exfoliating particles such as polyethylene and silica or alumina; cooling agents such as menthol; skin calming agents such as aloe vera; and colorants.

In addition, the compositions of the invention may further include 0.5 to 10% by weight of sequestering agents, such as tetra sodium ethylenediaminetetraacetate (EDTA), EHDP or mixtures; opacifiers and pearlizers such as ethylene glycol distearate, titanium dioxide or Lytron 621 (Styrene/Acrylate copolymer); all of which are useful in enhancing the appearance or properties of the product.

The composition may comprise water in amount of 10 to 95% by weight of the composition, more preferably from 25 to 90%, even more preferably from 32 to 85%, most preferably from 45 to 78% by weight of the composition.

Preferably, the composition has a viscosity of at least 10 mPa·s, more preferably in the range 30 to 10000 mPa·s, even more preferably 50 to 5000 mPas, and most preferably 100 to 2000 mPas, when measured at 20 degrees C. at a relatively high shear rate of about 20 s⁻¹. Preferably, the composition is in the form of fluid.

Preferably, the personal care composition is a skin care composition. The term “skin” as used herein includes the skin on the face, neck, chest, abdomen, back, arms, under arms, hands, and legs. Preferably “skin” means includes the skin on the face and under arms, more preferably skin means skin on the face other than lips and eyelids.

Without wishing to be bound by any specific theory or explanation, inflammation, a biological host response to harmful stimuli, is a mechanism by which the host removes the stimuli and initiates the healing process for self-protection. The innate immune system for a host is the first line of defense against invading organisms in a non-specific manner.

Dysregulated inflammation may cause various personal care problems including gingivitis/periodontitis (in the oral cavity), dandruff (on scalp/hair) and eczema acnes (on skin). Inflammation is a process that is manifest on the topical surface of the human or animal body in one or all the above described conditions.

Therefore, the present invention also provides a method of alleviating, inhibiting and/or reducing at least one symptom selected from inflammation, gingivitis, periodontitis, dandruff and acne, comprising a step of applying the personal care composition of the present invention. Preferably, the method is method of providing anti-inflammatory benefit, preferably of inhibiting release of at least one pro-inflammatory cytokines, particularly due to contact of skin with a pollutant.

In addition, the present invention also provides use of the personal care composition of the present invention for alleviating, inhibiting and/or reducing at least one symptom selected from inflammation, gingivitis, periodontitis, dandruff and acne. Preferably, the use is for providing anti-inflammatory benefit, more preferably for inhibiting release of at least one pro-inflammatory cytokines, particularly due to contact of skin with a pollutant.

It is preferred that the pollutant is a particulate pollutant. More preferably the particulate pollutant is at least one of squalene peroxide, PM_{2.5} or PM₁₀. Preferably the use is non-therapeutic. Preferably the method is non-therapeutic. The term non-therapeutic means for cosmetic purposes and not curative or therapeutic purposes. More preferably the pro-inflammatory cytokine is TNF-α, IL-8 or IL-1α.

The present invention also provides a personal care composition comprising extract of moringa; extract of plant of genus Theobroma; and 0.00001 to 0.08% of vitamin B3 compound by weight of the composition for use in the treatment of at least one symptom selected from inflammation, gingivitis, periodontitis, dandruff and acne, preferably for providing anti-inflammatory benefit, more preferably for inhibiting release of at least one pro-inflammatory cytokines, particularly due to contact of skin with a pollutant.

The present invention also provides use of extract of moringa; extract of plant of genus Theobroma; and 0.00001 to 0.08% of vitamin B3 compound by weight in the manufacture of a medicament for the treatment of at least one symptom selected from inflammation, gingivitis, periodontitis, dandruff and acne, preferably for providing anti-inflammatory benefit, more preferably for inhibiting release of at least one pro-inflammatory cytokines, particularly due to contact of skin with a pollutant.

The following examples are provided to facilitate an understanding of the invention. The examples are not intended to limit the scope of the claims.

### EXAMPLES

### Materials

| | | | | Active |
|---|---|---|---|---|
| Supplier | Trade name | Active | Abbreviation | wt % |
| BASF | Purisoft ® LS9726 ^{a} | Moringa seed | LS9726 | 1.6% |
| | | extract | | |
| Naturex SA | Moringa oil | Moringa | Moringa oil | 100% |
| | (LA030075- | refined | | |
| | refined) ^{b} | seed oil | | |
| Ashland | Blumilight ™ | Theobroma | Blumilight | 1% |
| | biofunctional ^{c} | cocoa | | |
| | | seed extract | | |
| Sigma | | Niacinamide | VB3 | >98.5% |
| Aldrich | | | | |

^{a} Purisoft ® LS9726 was procured from BASF Beauty Solutions France SAS.
^{b} Moringa oil (LA030075-refined) was procured from Naturex SA, France.
^{c} Blumilight ™ biofunctional was procured from Ashland (China) Investing Company.

### Example 1

EpiKutis® living skin equivalent (LSE), is a 3D reconstructed human epidermis model with Chinese human keratinocytes through air-liquid interface culture in a defined serum free medium, supplied by supplied by Biocell (Xi'an, China). Squalene monohydroperoxide (SQOOH), a stress surrogate, was prepared in house, by subjecting squalene to solar irradiation in the presence of a sensitizer.

The SQOOH was diluted using squalene. The moringa oil was dissolved in DMSO (dimethyl sulfoxide) at a concentration of 100 mM to form a stock solution. The samples in Table 1 were prepared accordingly by diluting the materials except moringa oil and/or stock solution with PBS (phosphate-buffered saline) to the desired levels as indicated in Table 1 before use for further experiment.

The sample was applied topically to the LSE followed by application of 3 μL of 1% SQOOH. The medium was then refreshed with interval of 24 hours. After 72 hours, the tissues were collected for histology and to test the extent of viability. The IL-1a expression was evaluated using a human immunoassay kit from R&D systems (Cat. DLA 50) and tested following the manufacturer's instructions.

The results were summarised in Table 1.

TABLE 1

| | | IL-1α level | Inhibition |
|---|---|---|---|
| Sample | Contained materials* | (pg/mL) | (%) |
| 1 | 1% of SQOOH (Control) | 8.8 ± 0.82 | — |
| 2 | 0.1% of LS9726 | 6.7 ± 0.59 | 24.20 |
| 3 | 0.02% of Moringa oil | 6.9 ± 0.60 | 21.18 |
| 4 | 0.01% of Blumilight | 8.3 ± 0.59 | 6.14 |
| 5 | 0.01% of VB3 | 6.6 ± 0.63 | 25.33 |
| 6 | 0.12% of VB3 | 5.8 ± 0.60 | 33.57 |
| 7 | 0.1% of LS9726 + | 3.7 ± 0.45** | 57.76 |
| | 0.02% of Moringa oil + | | |
| | 0.01% of Blumilight + | | |
| | 0.01% of VB3 | | |
| 8 | 0.1% of LS9726 + | 9.2 ± 1.10 | −4.54 |
| | 0.02% of Moringa oil + | | |
| | 0.01% of Blumilight + | | |
| | 0.12% of VB3 | | |

*The level refers to the level of the raw material, instead of active, in weight percentage.
**Significant different with any of 1,2,3,4,5,6 (p < 0.05)

The data in Table 1 indicates the IL-1a expression were inhibited significantly when including very low level of niacinamide into the combination of moringa extract and Theobroma cocoa seed extract.

1-12. (canceled)

13. A personal care composition comprising:
(a) extract of moringa;
(b) extract of plant of genus Theobroma; and
(c) 0.0001% to 0.06% by weight of vitamin B3 compound,
wherein the extract of moringa comprises Moringa Pterygosperma Seed Extract and Moringa Oleifera Seed Oil.

14. The composition according to claim 13 wherein the extract of moringa makes up from 0.000001 to 2% by weight of the composition.

15. The composition according to claim 13 wherein the extract of plant of genus Theobroma is selected from Theobroma Cacao (Cocoa) Husk Extract, Theobroma Cacao (Cocoa) Seed Extract, or a mixture thereof.

16. The composition according to claim 13 wherein the extract of plant of genus Theobroma makes up from 0.000001 to 1% by weight of the composition.

17. The composition according to claim 13 wherein the vitamin B3 compound is selected from niacin and niacinamide.

18. The composition according to claim 13 wherein the vitamin B3 compound makes up from 0.002% to 0.03% by weight of the composition.

19. The composition according to claim 13 wherein the extract of plant of genus Theobroma and the niacinamide are respectively present at a weight ratio from 1:1 to 30000:1.

20. A method of alleviating, inhibiting and/or reducing at least one symptom selected from inflammation, gingivitis, periodontitis, dandruff or acne or inhibiting release of pro-inflammatory cytokines or a combination thereof comprising a step of applying to skin the personal care composition of claim 13.

21. The method according to claim 20 wherein the composition after topical application inhibits release of pro-inflammatory cytokines.

22. The method according to claim 20 wherein the composition after topical application inhibits dandruff formation.

23. The method according to claim 20 wherein the composition after topical application inhibits acne formation.

24. The composition according to claim 13 wherein the composition further comprises soap, anionic surfactant, amphoteric surfactant, cationic surfactant, nonionic surfactant or a mixture thereof.

25. The composition according to claim 13 wherein the composition further comprises an antimicrobial agent.

26. The composition according to claim 13 wherein the composition further comprises betaine, linear alkylbenzene sulphonate, alkyl sulphate, alkyl ether sulphate, olefin sulphonate, alkyl xylene sulphonate, dialkyl sulphosuccinate, fatty acid ester sulphonate, salts thereof or a mixture thereof.
